(19) United States
(12) Patent Application Publication
(10) Pub. No.: US 2006/0009828 A1
(43) Pub. Date: Jan. 12, 2006

(54) Bipolar cardiac pacing wire with auxiliary pole

(76) Inventors: Marc Bemurat, Leognan (FR); Diane Bemurat, Leognan (FR); Laurent Bemurat, Leognan (FR); Eliane Bemurat, Leognan (FR)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/120,380

(22) Filed: May 3, 2005

(30) Foreign Application Priority Data
May 5, 2004 (FR) 04 04850

(51) Int. Cl. A61N 1/05

(52) U.S. Cl. 607/119

(57) ABSTRACT

The object of the invention is a bipolar cardiac pacing wire with colinearly wound leads and an auxiliary pole, characterized in that said auxiliary pole (P_{A}) is connected to one of two connectors (D, P) that connect wire (1) to the casing of the pacemaker by a third lead colinearly wound with the two leads of the wire.

### Related Application

The present application claims priority to French Application No. 04 04850 filed May 5, 2004.

### TECHNICAL FIELD

This invention relates to bipolar cardiac pacing wires having an external temporary auxiliary pacing pole that allows the casing of the pacemaker to be replaced without the necessity of interrupting the pacing of the heart.

### SUMMARY OF THE INVENTION

The goal of the invention is to simplify the design of cardiac pacing wires and to make access to the auxiliary pacing pole easier and more efficient.

To this end, the object of the invention is a bipolar cardiac pacing wire with colinearly coiled leads and with an auxiliary pole, the auxiliary pole being connected to one of the two connectors linking the wire to the pacemaker casing by a third lead colinearly coiled with the two leads of the wire.

This colinear arrangement of the three leads of the wire is an elegant solution to the placement of the three leads and their connections, especially as regards the lead assigned to the auxiliary pacing pole which is shunted to the most suitable location of the wire, for example to the junction between the cord of the wire and the tip of the connection to the pacemaker casing.

The pacing wire maintains a reduced diameter due to the colinearity of the three leads over the entirety of the common path, and it has high reliability.

The auxiliary lead is preferably connected to the wire connector, which is coupled to the distal pacing electrode, but it could be connected to the other connector coupled to the proximal electrode.

The invention likewise relates to structures of the auxiliary pacing pole that are suited to this new type of pacing wire.

Other characteristics and advantages will become apparent from the following description of embodiments of the invention, given by way of example only and with reference to the attached drawings.

### BRIEF DESCRIPTION OF THE DRAWINGS

FIG. 1 is a schematic diagram depicting the pacing wire in accordance with the invention;

FIG. 2 depicts the manner in which the distal electrode of the lead of the auxiliary pole and one of the two leads of the pacing wire are connected to the connector;

FIG. 3 depicts an alternative connection of the lead of the auxiliary pole;

FIG. 4 depicts the branching zone of the wire with its auxiliary pole;

FIG. 5 depicts how the wire in use;

FIG. 6 depicts a first embodiment of the auxiliary pole; and

FIGS. 7 and 8 depict a second embodiment of the auxiliary pole.

### DETAILED DESCRIPTION OF THE DRAWINGS

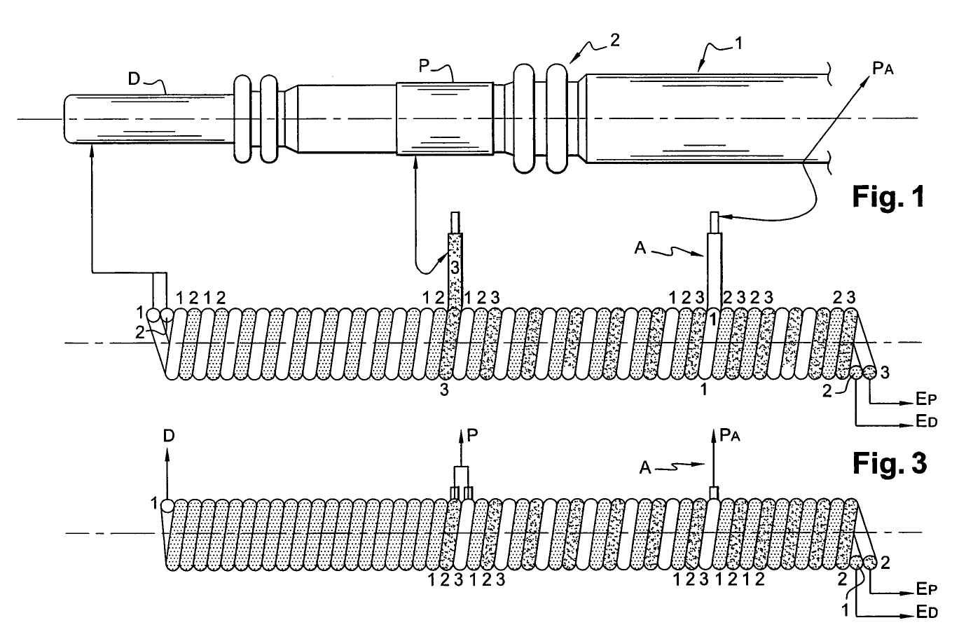

In FIG. 1, a bipolar pacing wire in accordance with the invention is annotated 1, with its tip 2 for connection to the pacemaker casing (not shown), showing a first connector D for connecting the pacemaker to distal electrode E_{D} of the wire and a second connector P for connection to proximal electrode E_{P}.

The two leads of wire 1 are conventional and are of the colinear type, that is, two leads having the same diameter wound in closed interleaved coil.

For this reason the leads are composed of a central conductive core, wrapped in an insulating sheath, the colinearity making it possible to significantly reduce the diameter of wire 1, for example, to 1.7 mm instead of 2.3 mm for conventional wires with two spiral coaxial leads of different diameters.

FIG. 1 shows three colinear leads labeled 1, 2 and 3 respectively. Lead no. 1 is the lead of the auxiliary pole and is thus electrically connected to connector D in the embodiment of FIG. 1. The other end of conductor no. 1 emerges from the common winding and is insulated at A, this end A being electrically connected to auxiliary pole P_{A} (not shown in FIG. 1).

Lead no. 2 is likewise electrically connected to connector D; however, its other end will be connected to distal electrode E_{D}, as shown in FIG. 1.

Lead no. 3 is the lead of the wire connecting connector P to proximal electrode E_{P}.

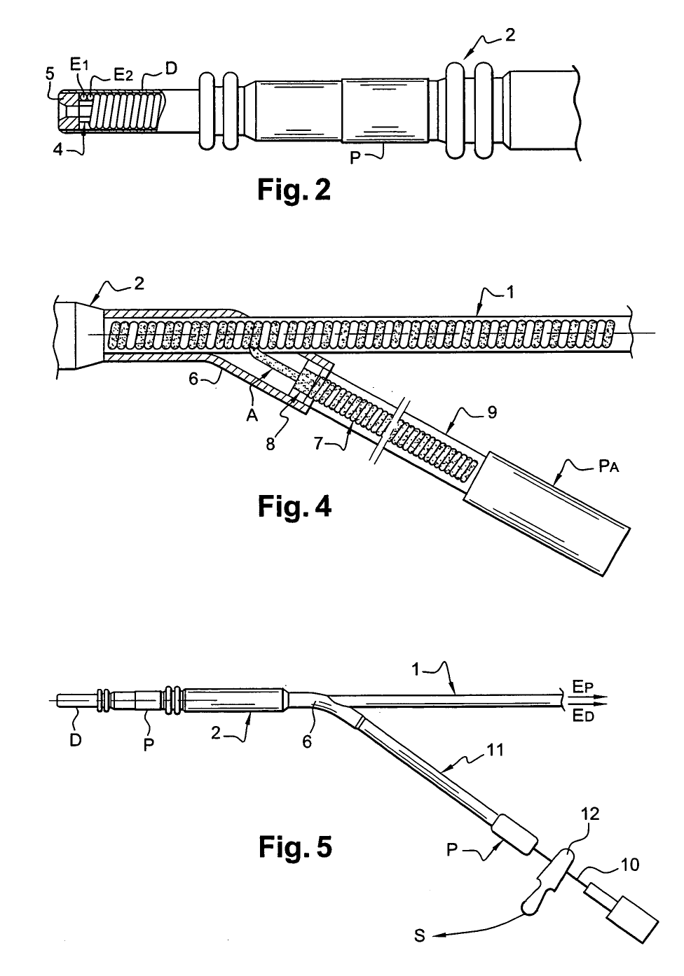

FIG. 2 shows one mode of electrical connection between connector D composed of a metal tubular sleeve, within which two colinear leads no. 1 and no. 2 are located, and of which ends E₁ and E₂ are soldered to a supported sleeve fitted in the internal space to the leads, and whose head 5 closes the end of sleeve D, the head being laser-welded to the sleeve.

FIG. 3 shows a version of the connection of auxiliary lead (no. 3), which is connected by one end to connector P and by the other end to auxiliary pole P_{A}.

Connector D is always connected to distal electrode E_{D} via lead no. 1 and lead P is connected to proximal electrode E_{P} via lead no. 2.

Thus the colinear coils have, in the two connection modes of FIGS. 1 and 3, a section which is common to the three leads between connector P and outlet point A of the branch connector, and a section common to the two leads between said output A and the electrodes of the pacing wire. Conversely, in the zone between connectors D and P, there are two leads (nos. 1 and 2) in FIG. 1 for a single lead (no. 1) in FIG. 3.

FIG. 4 shows the branching of the wire routing the branching lead (output A) to auxiliary pacing pole P_{A}. The branching is achieved at the junction of the cord of the wire to tip 2 for connection to the casing and is implemented by a sleeve 6 made of insulating material such as silicone.

The electrical connection between end A of auxiliary lead and pole P_{A} is implemented by a stripped spiral lead 7, connected to end A by a connecting conductive plug 8 and covered by a silicone insulating sheath 9.

FIG. 5 shows how auxiliary pole P_{A} is used. A conductive stylet 10 is inserted into pole P_{A} so as to come into contact with lead 7 of branch 11. An alligator clip 12 for example is attached to stylet 10 and is electrically connected to an external pacemaker 5 (not shown).

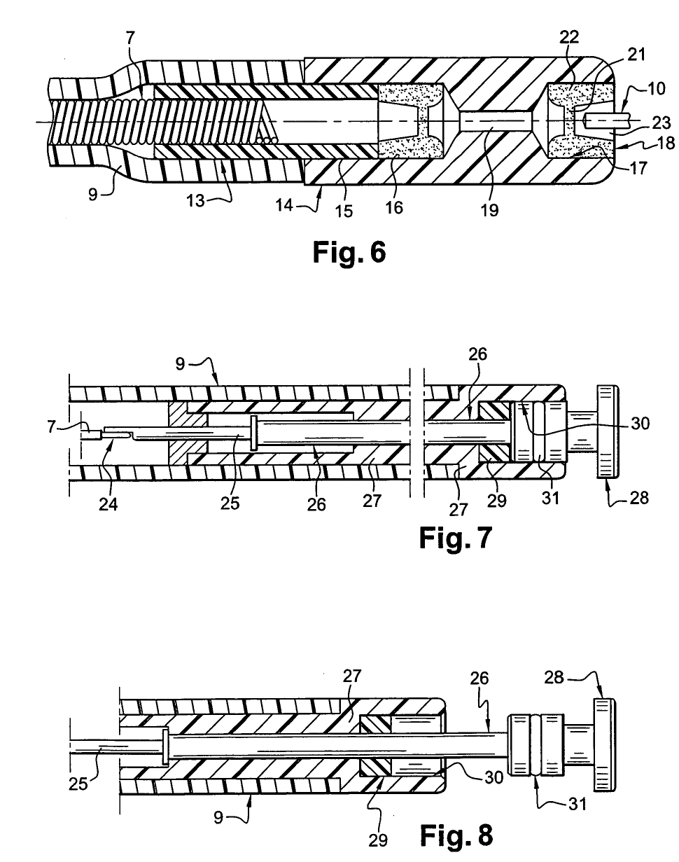

FIG. 6 shows a first embodiment of auxiliary pole P_{A}. The end of lead 7 is forcibly inserted at least partially into a sleeve 13 of electrically insulating material, which is itself partially inserted into a second sleeve 14 of electrically insulating material.

Sleeve 14 on one end includes a cylindrical housing 15 partially occupied by sleeve 13, the remainder of the housing being occupied by a closure member 16 of silicone or the like. The other end of sleeve 15 defines a cylindrical housing 17 which holds a closure member 18 made of silicone or the like which is identical to closure member 16.

Between two housings 15 and 17 a conduit 19 of reduced diameter is arranged coaxially to them.

Closure members 16 and 18 have a shape that facilitates the insertion of stylet 10 of FIG. 5.

Input closure member 18 has a central shell 21, which is surrounded by a more massive annular part 22 that defines a cavity 23 with the shape of a truncated cone for guiding the insertion of stylet 10 in the direction of the central shell 21 having a diameter slightly greater than that of guide channel 19. Shell 21 can be pre-perforated or pre-split.

Thus, the penetration of stylet 10 into connecting tip 14 is perfectly guided and the end of the stylet is perfectly centered on the axis of lead 7.

Stylet 10 is fitted to the interior of the lead 7 over a certain distance and is kept perfectly stationary relative to the tip by the pressure of the material of the two closure members 16 and 18 that are traversed. Thus the physical contact between the stylet and the lead is guaranteed to be reliable and effective in electrical terms, while having an optimum seal at the level of tip 14 due to double closure members (16, 18).

FIGS. 7 and 8 show a second embodiment of auxiliary pole P_{A}.

The end (auxiliary pole side) of branching lead 7 is shown at 24.

This end 24 is soldered to a stainless steel wire 25 on which a likewise stainless steel tube 26 can slide, the latter itself mounted so as to be able to slide in an insulating sleeve 27 to which the insulating sheath 9 is welded.

Tube 26 can be actuated using a knob 28, which is integral with tube 26.

A wiper seal housed in a chamber 30 installed on the end of sleeve 27 is shown at 29 and an O-ring which is placed between tube 26 and knob 28 and which is able to slide into chamber 30 is shown at 31. This device ensures the seal of the auxiliary pole when knob 28 is in the pushed-in position (FIG. 7) which blocks chamber 30.

FIG. 8 shows knob 28 in the output position, thus exposing the part of tube 26 on which alligator clip 12 of FIG. 5 can be attached.

The invention is not limited to the illustrated example and can be applied especially to a pacing wire for which an adapter is inserted either between tip 2 and the casing of the pacemaker or between wire 1 and the implanted proximal and distal electrodes.

1. A bipolar cardiac pacing wire for a pacemaker having a casing, the pacing wire comprising:
first and second leads colinearly wound;
first and second wire connectors connecting the pacing wire to the casing; and
an auxiliary pole having an auxiliary lead colinearly wound with the first and second leads, wherein the auxiliary pole is connected to one of the first and second wire connectors by the auxiliary lead.

2. A pacing wire as claimed in claim 1, further comprising a distal pacing electrode coupled to the first wire connector, wherein the auxiliary lead is connected to the first wire connector.

3. A pacing wire as claimed in claim 1, further comprising an auxiliary conductor having an output, a stripped spiral portion, and an insulator, wherein the auxiliary pole is connected to the output by the stripped spiral portion and the insulator sheaths the stripped spiral portion.

4. A pacing wire as claimed in claim 3, further comprising a tip connecting the pacing wire to the casing, wherein the output of the auxiliary conductor is proximate the tip.

5. A pacing wire as claimed in claim 4, further comprising a branching zone and an insulating sleeve, wherein the insulating sleeve covers the branching zone.

6. A pacing wire as claimed in claim 3, the pacemaker further comprising an auxiliary pacing supply source having a stylet for electrical connection of the auxiliary lead to the auxiliary pacing supply source;
the auxiliary pole comprising an electrically insulating sleeve having two housings holding silicone closure members, the housings arranged coaxially to the spiral connector;
the auxiliary pole further comprising an interior and an axial conduit separating the housings, the axial conduit having a reduced diameter for guiding the stylet into the interior of the auxiliary pole, such that contact of auxiliary lead and the auxiliary pacing supply source is made by insertion of the stylet into the interior of the auxiliary pole.

7. A pacing wire as claimed in claim 3, the pacemaker further comprising an external pacing supply source;
the pacing wire further comprising a conductive wire operably connected to the spiral conductor;
the auxiliary pole comprising an insulating sleeve and a conductive tube having a knob, the conductive tube movable by the knob and slidable relative to the conductive wire, wherein the conductive tube is mounted to slide inside the insulating sleeve, such that the conductive tube is exteriorly accessible for connection to the external pacing supply source.

8. A pacing wire as claimed in claim 7, further comprising a sealing device disposed between the knob and the insulating sleeve, wherein when the knob is disposed inwardly, the conductive tube is exteriorly insulated by the sealing device.
